(19) United States
(12) Patent Application Publication
(10) Pub. No.: US 2014/0210987 A1
(43) Pub. Date: Jul. 31, 2014

(54) Temperature Display Helmet

(71) Applicant: Chad Adkins, Portland, TN (US)

(72) Inventor: Chad Adkins, Portland, TN (US)

(21) Appl. No.: 14/107,353

(22) Filed: Dec. 16, 2013

Related U.S. Application Data
(60) Provisional application No. 61/758,768, filed on Jan. 31, 2013.

(51) Int. Cl. H04N 5/33

(52) U.S. Cl. CPC H04N 5/33
USPC 348/82; 348/164

(57) ABSTRACT

A heads-up digital display within a firefighting or welding helmet that indicates the temperature of the observed region is disclosed. Data is provided by a thermal imaging camera mounted on or through the helmet's exterior surface. Readings of the temperature across the field of vision may be localized and calibrated to a target point such as crosshairs, printed on the face shield, or may be generalized, for example taking an average temperature or maximum temperature from across the field of vision. The temperature reading would be displayed digitally on a temperature display mounted within the helmet.

### CROSS-REFERENCE TO RELATED APPLICATIONS

This application claims priority to U.S. Provisional Application No. 61/758,768, filed Jan. 31, 2013, which is hereby incorporated by reference.

### STATEMENT REGARDING FEDERALLY SPONSORED RESEARCH OR DEVELOPMENT

Not Applicable

### PARTIES TO A JOINT RESEARCH AGREEMENT

Not Applicable

### REFERENCE TO SEQUENCE LISTING, A TABLE, OR A COMPUTER PROGRAM LISTING COMPACT DISK APPENDIX

Not Applicable

### BACKGROUND OF THE INVENTION

The invention relates generally to helmets, and in particular to a heads-up digital display within a helmet, such as a welding or firefighter's helmet, that indicates the temperature of the observed region. When welding or performing other metal fabrication tasks, welders and machinists frequently need to know the temperature of the metal parts they are fabricating, to produce welds of optimum strength. Also, firefighters often need to know the temperature in areas where they are working to reduce the risk of injury. A helmet that can provide a digital temperature reading of the metal being worked upon, or a specific area where a firefighter is working, would be of immense value in saving time and material, and reducing the risk of serious injury, for both welders and firefighters. Data is provided by a thermal imaging camera mounted on or through the helmet exterior. Readings may be localized and calibrated to a target point (e.g. crosshairs) printed on the helmet, or may be generalized, for example taking an average temperature or maximum temperature from across the field of vision. Alternatively, a thermal image replicating the field of view in false color may be displayed.

Previous examples of helmets having temperature sensor capability are known the prior art. Welding and other helmets are known that attempt to assess the weld temperature or cooling rate, both by direct sensing and by monitoring of proxy data, such as the weld time and welding torch output. The prior art lacks, however, a helmet employing a thermal imaging camera that outputs the precise temperature over a precisely specified and localized region the user's field of view.

### SUMMARY OF THE INVENTION

Accordingly, the invention is directed to a heads-up digital display within a firefighting or welding helmet that indicates the temperature of the observed region. Data is provided by a thermal imaging camera mounted on or through the helmet's exterior surface. Readings of the temperature across the field of vision may be localized and calibrated to a target point such as crosshairs, printed on the face shield, or may be generalized, for example taking an average temperature or maximum temperature from across the field of vision. The temperature reading would be displayed digitally on a temperature display within the helmet.

Additional features and advantages of the invention will be set forth in the description which follows, and will be apparent from the description, or may be learned by practice of the invention. The foregoing general description and the following detailed description are exemplary and explanatory and are intended to provide further explanation of the invention.

### BRIEF DESCRIPTION OF THE DRAWINGS

The accompanying drawings are included to provide a further understanding of the invention and are incorporated into and constitute a part of the specification. They illustrate one embodiment of the invention and, together with the description, serve to explain the principles of the invention.

FIG. 1 shows an exterior perspective view of the first exemplary embodiment displaying the helmet 10, the face shield 11, the viewing pane 11A, the crosshairs 12, the strap 13, and the thermal imaging camera 14.

FIG. 2 shows an interior view of the first exemplary embodiment displaying the helmet 10, the face shield 11, the viewing pane 11A, the crosshairs 12, the strap 13, the temperature display 15, and the cable 20.

FIG. 3 shows a cross-sectional view of the first exemplary embodiment displaying the helmet 10, the face shield 11, the viewing pane 11A, the thermal imaging camera 14, the temperature display 15, and the cable 20.

FIG. 4 shows an interior view of the second exemplary embodiment displaying the firefighting mask 40, viewing pane 41, crosshairs 42, thermal imaging camera 44, temperature display 45, and cable 46.

FIG. 5 shows a front perspective view of a the third exemplary embodiment, displaying the firefighter's hat 50, sensor unit 51, thermal imaging camera 54, temperature display 55, and cable 56.

FIG. 6 shows a front perspective view of the sensor unit of the third exemplary embodiment, displaying the sensor unit 51, thermal imaging camera 54, battery compartment 60, and on/off switch 61.

### DETAILED DESCRIPTION OF THE INVENTION

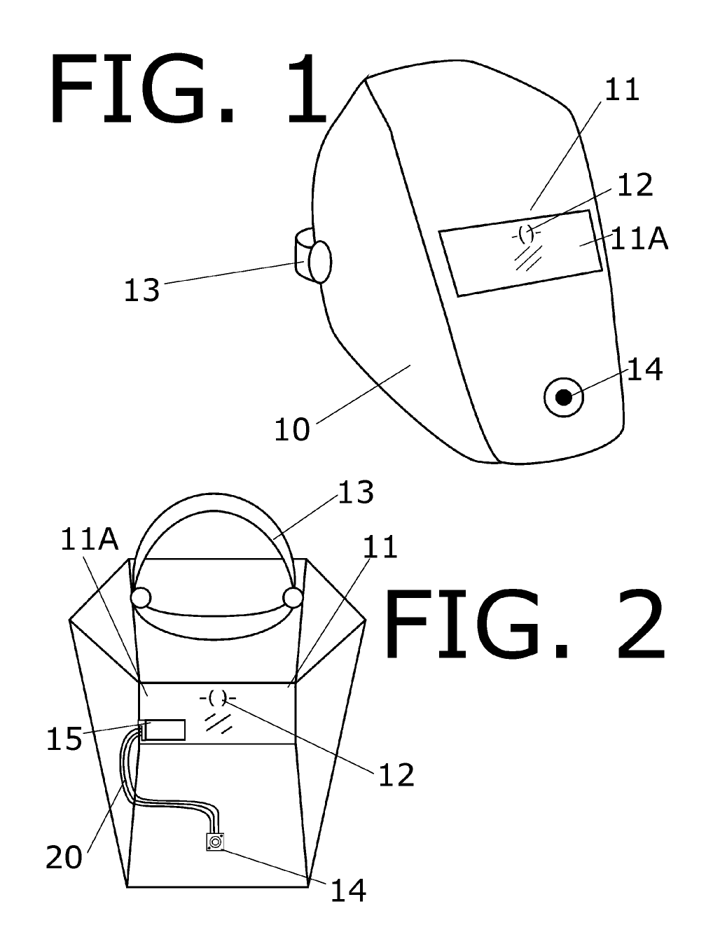

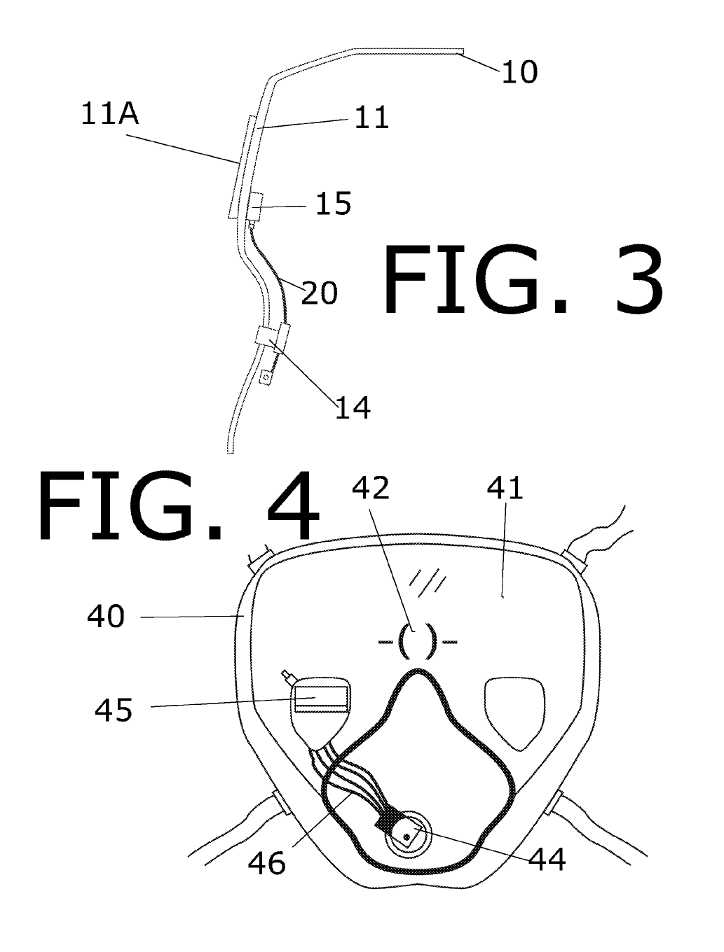

Referring now to the invention in more detail, the invention in its first exemplary embodiment of FIGS. 1-3 is directed to a heads-up digital display within a helmet 10, for example a welding-style helmet, that indicates the temperature of the observed region. The helmet 10 is secured to the user's head with a strap 13 and has a moveable or non-moveable face shield 11 with a transparent viewing pane 11A. Data is provided by a thermal imaging camera 14 mounted on or through the exterior of the helmet 10. Readings from the thermal imaging camera 14 may be localized and calibrated to a target point such as crosshairs 12 printed on the viewing pane 11A, or may be generalized, for example taking an average temperature or maximum temperature from across the field of vision or a portion thereof. A numeric temperature reading would be displayed digitally on the interior surface of the face shield 11 by a temperature display 15.

Electronic components include an infrared thermal imaging camera 14 and a processor. The thermal imaging camera 14 contains an array of infrared detectors, which receive thermal infrared images from the area being viewed, centered on the crosshairs 12, and transmit them to the processor, which then transmits the temperature of the field of vision or single object to the temperature display 15. Power and data may be transmitted between the thermal imaging camera 14 and the temperature display 15 via a cable 20. The temperature display may be a self-contained liquid crystal display screen as shown or may be a display embedded within or projected upon the viewing pane 11A.

The thermal imaging camera 14 is thermally insulated from the field in which the readings are taken, ensuring optimal performance. The processor applies emissivity algorithms (such algorithms being well-known in the prior art) to determine the true temperature of the object being viewed based on its thermal radiation emission. Cooled and uncooled infrared detectors are contemplated. Cooled infrared detectors would contain the necessary cooling components. Due to the weight of cooled infrared detectors, an uncooled infrared detector is preferable.

Overall size of the helmet 10 would preferably—be twelve inches in length and nine inches wide, to adequately cover the face and throat of the user. The helmet 10 would be manufactured from lightweight, heat-resistant material such as thermoplastic. The face shield 12 would be manufactured from transparent, highly heat-resistant material such as thermal glass, optionally with reduced optical transmission to protect the eyes of the user from high luminosity light and flame.

Referring now to the second exemplary embodiment of FIG. 4, a firefighting mask, the second exemplary embodiment provides a firefighting mask 40 having a viewing pane 41. Printed on the viewing pane 41 may be a crosshairs 12. Mounted on or through the mask interior is a temperature display 45 linked via a cable 46 to a thermal imaging camera 44. The components of the second exemplary embodiment operate similarly to those of the first exemplary embodiment, except insofar as the materials and shapes used are optimized for a firefighting environment as opposed to other thermal environments, for example a welding environment.

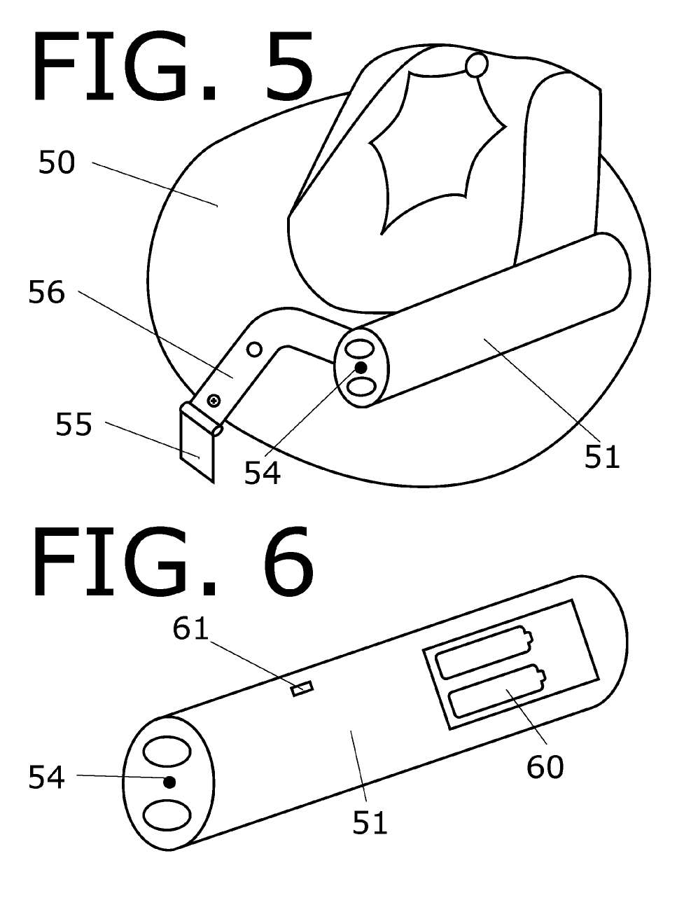

Referring now to the third exemplary embodiment of FIGS. 5-6, a firefighter's hat, the third exemplary embodiment provides a firefighter's hat 50 having a sensor unit 51 mounted thereon or therethrough. The sensor unit may be linked to a temperature display 55 via a cable 56. The sensor unit 51 would feature a thermal imaging camera 54, battery compartment 60 (shown in FIG. 6 with a battery compartment cover removed), and on/off switch 61. The components of the third exemplary embodiment operate similarly to those of the first exemplary embodiment, except insofar as the materials and shapes used are optimized for a firefighting environment as opposed to other thermal environments, for example a welding environment.

Component sizes and materials listed above are preferable, but artisans will recognize that larger or smaller components, or different materials, could be selected without altering the scope of the invention. The device would be manufactured from a rigid, heat resistant material such as bamboo, teakwood or injection molded plastic.

While the foregoing written description of the invention enables one of ordinary skill to make and use what is presently considered to be the best mode thereof, those of ordinary skill in the art will understand and appreciate the existence of variations, combinations, and equivalents of the specific embodiment, method, and examples herein. The invention should, therefore, not be limited by the above described embodiment, method, and examples, but by all embodiments and methods within the scope and spirit of the invention.

I claim:

1. An article of headwear to be worn by a user comprising:
(a) a head or face covering for covering the head or face of said user;
(b) said head or face covering being adapted for use in the presence of any of the group of welding equipment, fire, electrical discharge, or hot objects;
(b) a digital display;
(c) said digital display being affixed to, within, or through said head or face covering for covering the head or face of said user such that said digital display is visible to the user;
(d) a thermal imaging camera;
(e) said thermal imaging camera being affixed to, within, or through said head or face covering for covering the head or face of said user;
(f) a power source;
(g) said power source being affixed, within, or through said head or face covering for covering the head or face of said user;
(h) said power source being in electronic communication with said digital display and said thermal imaging camera such that said power source provides power to said digital display and to said thermal imaging camera;
(i) an output signal;
(j) said output signal being generated by said thermal imaging camera;
(k) said thermal imaging camera being in electronic communication with said digital display such that said output signal is transmitted from said thermal imaging camera to said digital display;
(l) said output signal comprising at least one numeric temperature value or image representing all of or a portion of the field of view of said thermal imaging camera; and
whereby said user may be informed of the thermal properties of the environment.

2. The article of headwear of claim 1 wherein said at least one temperature value comprises any of the group of:
(a) a numeric value representing the temperature of a designated point within the field of view of said thermal imaging camera;
(b) a numeric value representing the average temperature over a localized region within the field of view of said thermal imaging camera;
(c) a numeric value representing the maximum temperature within a localized region within the field of view of said thermal imaging camera;
(d) a numeric value representing the average temperature over the entire field of view of said thermal imaging camera; and
(e) a numeric value representing the maximum temperature within the entire field of view of said thermal imaging camera.

3. The article of headwear of claim 2 wherein:
(a) said head or face covering for covering the head or face of said user comprises a face covering region for covering the face of the user;
(b) said face covering region for covering the face of the user comprising a transparent viewing pane;
(c) a crosshairs;
(d) said crosshairs being applied to said transparent viewing pane such that said crosshairs defines a target point relative to the field of view of said user; and
(e) any of said at least one temperature value being defined relative to said target point.

4. The article of headwear of claim 1 wherein said head or face covering for covering the head or face of said user comprises a face covering region for covering the face of the user is in the style of a welding helmet.

5. The article of headwear of claim 2 wherein said head or face covering for covering the head or face of said user comprises a face covering region for covering the face of the user is in the style of a welding helmet.

6. The article of headwear of claim 3 wherein said head or face covering for covering the head or face of said user comprises a face covering region for covering the face of the user is in the style of a welding helmet.

7. The article of headwear of claim 1 wherein said head or face covering for covering the head or face of said user comprises a face covering region for covering the face of the user is in the style of a firefighting mask.

8. The article of headwear of claim 2 wherein said head or face covering for covering the head or face of said user comprises a face covering region for covering the face of the user is in the style of a firefighting mask.

9. The article of headwear of claim 3 wherein said head or face covering for covering the head or face of said user comprises a face covering region for covering the face of the user is in the style of a firefighting mask.

10. The article of headwear of claim 1 wherein said head or face covering for covering the head or face of said user comprises a face covering region for covering the face of the user is in the style of a firefighter's hat.

11. The article of headwear of claim 2 wherein said head or face covering for covering the head or face of said user comprises a face covering region for covering the face of the user is in the style of a firefighter's hat.

12. The article of headwear of claim 10 wherein said thermal imaging camera and said power source are contained within a sensor unit, said sensor unit being affixed to the exterior of said head or face covering for covering the head or face of said user.

13. The article of headwear of claim 11 wherein said thermal imaging camera and said power source are contained within a sensor unit, said sensor unit being affixed to the exterior of said head or face covering for covering the head or face of said user.

14. The article of headwear of claim 10 wherein said digital display is positioned on said head or face covering for covering the head or face of said user such that said digital display extends down from said head or face covering for covering the head or face of said user into the field of view of said user.

15. The article of headwear of claim 11 wherein said digital display is positioned on said head or face covering for covering the head or face of said user such that said digital display extends down from said head or face covering for covering the head or face of said user into the field of view of said user.

16. The article of headwear of claim 12 wherein said digital display is positioned on said head or face covering for covering the head or face of said user such that said digital display extends down from said head or face covering for covering the head or face of said user into the field of view of said user.

17. The article of headwear of claim 13 wherein said digital display is positioned on said head or face covering for covering the head or face of said user such that said digital display extends down from said head or face covering for covering the head or face of said user into the field of view of said user.
